(19) United States
(12) Patent Application Publication
(10) Pub. No.: US 2025/0312300 A1
(43) Pub. Date: Oct. 9, 2025

(54) BRIDGED BICYCLIC CARBOXYLIC ACIDS FOR TREATING OSTEOSARCOMA AND EWING SARCOMA

(71) Applicant: UEA ENTERPRISES LIMITED, Norwich Norfolk (GB)

(72) Inventors: Darrell GREEN, Norwich Norfolk (GB); William FRASER, Norwich Norfolk (GB)

(73) Assignee: UEA ENTERPRISES LIMITED, Norwich Norfolk (GB)

(21) Appl. No.: 18/860,392

(22) PCT Filed: Apr. 27, 2023
(86) PCT No.: PCT/EP2023/061092

§ 371 Date: Oct. 25, 2024

(30) Foreign Application Priority Data
Apr. 28, 2022 (GB) 2206183.2

(51) Int. Cl. A61K 31/192; A61P 35/00

(52) U.S. Cl. CPC A61K 31/192; A61P 35/00

(57) ABSTRACT

The present invention provides a composition for use in a method of treating osteosarcoma, Ewing sarcoma and/or a metastatic cancer originating from osteosarcoma or Ewing sarcoma. The present invention also provides a method of treating a cancer selected from the group consisting of osteosarcoma, Ewing sarcoma, a metastatic cancer originating from an osteosarcoma and a metastatic cancer originating from Ewing sarcoma.

### BACKGROUND

An estimated 1,095 children are diagnosed with cancer every day worldwide according to the World Health Organisation. According to Public Health England's children, teenagers and young adults UK cancer statistics report 2021, there were 260 childhood cancer deaths, accounting for 7% of all childhood deaths (0-14-year-olds). For teenagers and young adults (15-24-year-olds) there were 290 cancer deaths, accounting for 11% of all teenager and young adults' deaths. Cancer remains the most common cause of childhood death outside of infancy, and the most common disease-related cause of death in teenagers and young adults: only accidents and suicide are responsible for more deaths in this age group. Similarly, childhood cancer is the leading cause of death by disease in children in the US.

Sarcomas (bone and soft tissue cancers) are the third commonest childhood cancers. Almost half of all sarcomas are primary bone cancer (PBC) affecting ˜6 per 10⁶ individuals per year. In the UK there are around 550 new PBC cases every year (Cancer Research UK), while in the US there are around 3,450 new cases of primary bone cancer every year (estimate from 2018, National Cancer Institute). PBC is heterogeneous with major subtypes underpinned by distinct genetic drivers leading to diverse morphological features and clinical behaviour, requiring significantly different treatment approaches. The two most common PBC subtypes in children are osteosarcoma (OS) and Ewing sarcoma (ES). The average age of patients with ES is 15-years old, while OS is most frequently found in older children, teenagers and young adults between the ages of 10 to 24 (Bone Cancer Research Trust).

Both OS and ES are high-grade at diagnosis and harbour an accelerated propensity for metastasis. 25% of patients present with detectable lung/bone metastases. Half of apparent localised cases harbour undetectable micrometastases that relapse later. Five-year survival for localised OS and ES is ˜50%, and for metastatic/relapse cases it is only ˜15%.

Current treatment involves non-specific combination chemotherapy (such as methotrexate, doxorubicin and cisplatin in OS; and vincristine, doxorubicin and cyclophosphamide alternating with ifosfamide and etoposide in ES) with surgery.

Surgery is highly invasive, sometimes involving whole limb amputation and still risks incomplete removal of the affected tissue. Other side effects of surgery are the risk of embolism and infection, as well as the potential need for further surgical treatment (known as revision surgery) and associated effects on the mental health of the patients due to the invasive procedures. Chemotherapy is associated with complex and severe side effects (and late effects) because it targets rapidly dividing cells all over the body. As children are still growing and many kinds of healthy cells are dividing faster than in adults, chemotherapy during childhood can damage these cells leading to long-term health defects on growth and development (American Cancer Society). Other side effects of chemotherapy include a lowered immune status leading to infections, nausea, vomiting, hair loss, loss of normal tissue and organ toxicity. Unfortunately, neither OS nor ES have seen treatment or survival improvement for decades. Therefore, new treatment options for ES and OS are urgently needed but the aggressive phenotype of ES and OS has hampered patient-benefitting tangible progress thus far when compared to other malignancies.

### SUMMARY OF INVENTION

The transcription factor RUNX family transcription factor 2 (RUNX2), is implicated in cancers commonly identified in adults. The present inventors have surprisingly found that inhibition of the activity and/or function of the RUNX2 protein, rather than aiming to affect the transcription or the translation of RUNX2, can also be of value in the treatment of the childhood cancers OS and ES.

The present invention provides a composition for use in a method of treating osteosarcoma, Ewing sarcoma and/or a metastatic cancer originating from osteosarcoma or Ewing sarcoma, wherein the composition comprises a compound according to formula (I) or a pharmaceutically acceptable salt, ester, derivative or prodrug thereof,

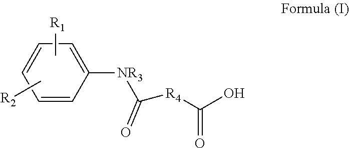

wherein R₁ and R₂ are each independently selected from hydrogen, a halogen, a haloalkyl, an alkyl, an alkylamide, a cycloalkylamide or an alkyamine; R₃ is H or alkyl; and R₄ is a bridged cycloalkenyl ring.

Preferably, the composition for the use of the present invention inhibits metastasis of the osteosarcoma or Ewing sarcoma. The composition for the use of the present invention therefore reduces metastasis of the osteosarcoma or Ewing sarcoma.

Advantageously, the osteosarcoma or Ewing sarcoma overexpresses RUNX2 in comparison to normal bone tissue or other biological material.

Optionally, the composition for the use of the present invention is a compound of formula (I) which is 3-(N-(3,4-dichlorophenyl)carbamoyl)-5-norbornene-2-carboxylic acid, also named 3-{[(3,4-dichlorophenyl)amino]carbonyl}bicyclo[2.2.1]hept-5-ene-2-carboxylic acid, known as CADD522

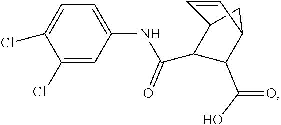 [SMILES: O=C(O)C1C2C=CC(C2)C1C(=O)Nc1ccc(Cl)c(Cl)c1]

or a salt, ester, derivative or prodrug thereof.

The present invention also provides a method of treating a cancer selected from the group consisting of osteosarcoma, Ewing sarcoma, a metastatic cancer originating from an osteosarcoma and a metastatic cancer originating from Ewing sarcoma, the method comprising administering to a subject in need thereof a composition comprising a compound according to formula (I) or a pharmaceutically acceptable salt, ester, derivative or prodrug thereof,

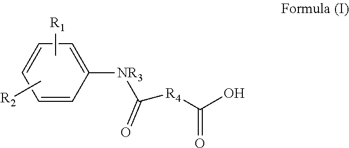

wherein R₁ and R₂ are each independently selected from hydrogen, a halogen, a haloalkyl, an alkyl, an alkylamide, a cycloalkylamide or an alkyamine; R₃ is H or alkyl; and R₄ is a bridged cycloalkenyl ring.

Preferably in the method of the present invention metastasis of the osteosarcoma or Ewing sarcoma is inhibited.

Advantageously, the osteosarcoma or Ewing sarcoma overexpresses RUNX2 in comparison to normal bone tissue or other biological material.

Optionally, the method of the present invention provides a compound of formula (I) which is 3-(N-(3,4-dichlorophenyl)carbamoyl)-5-norbornene-2-carboxylic acid, also named 3-{[(3,4-dichlorophenyl)amino]carbonyl}bicyclo[2.2.1]hept-5-ene-2-carboxylic acid, known as CADD522

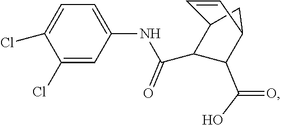 [SMILES: O=C(O)C1C2C=CC(C2)C1C(=O)Nc1ccc(Cl)c(Cl)c1]

or a salt, ester, derivative or prodrug thereof.

The present invention has one or more of the following advantages over and above the prior art.

The invention provides an alternative treatment for OS and ES.

The invention provides an improved treatment for OS and ES.

Current treatment methods for OS and ES are highly invasive (surgery) and may have severe short-term and long-term side effects, especially in children (chemotherapy). Identification of alternative compounds for use in treatments and alternative treatment methods has been very difficult due to OS and ES being high-grade at diagnosis. Surprisingly and unexpectedly, the composition of the present invention was found to be effective against OS and ES tumour cells in vitro and against OS and ES tumours in vivo. This provides, for the first time, a small molecule treatment of OS and ES as an alternative to cytotoxic chemotherapy.

As described above chemotherapy targets all rapidly dividing cells. In contrast, the present invention provides a composition for treating OS and ES, which specifically targets cancer cells with aberrant expression and activity of the OS/ES marker RUNX2. This may lead to reduced side effects and an enhanced safety profile as compared to chemotherapy.

Five-year survival for localised OS and ES is ˜50% but for metastatic/relapse cases it is only ˜15% and hence patients have very poor prognosis. Advantageously, the composition of the invention was found to specifically increase metastasis free survival in in vivo mice experiments discussed below. Therefore, the present invention provides a highly promising treatment option effective to prevent metastasis, and/or treat metastatic OS and ES.

Importantly, the invention also provides an additional treatment option for OS and ES, which may be used in combination with chemotherapy and/or surgery. Therefore, the composition of the invention may also be used for targeted treatment of any remaining OS or ES cells after resection of the tumour by surgery, and/or in combination with chemotherapy.

### BRIEF DESCRIPTION OF FIGURES

FIG. 1 shows OS cell (143B(OS)-GFP-Luc) viability/proliferation in response to CADD522 treatment in vitro after 72 h. Notably, addition of CADD522 at a concentration of 100 μM has a cytotoxic effect.

FIG. 2 shows ES cell (TC71(ES)-GFP-Luc) viability/proliferation in response to CADD522 treatment in vitro after 72 h.

FIG. 3 shows OS tumour volume over time in control vs. CADD522 treated mice (p=0.0072) (left hand figure) and ES tumour volume over time in control vs. CADD522 treated mice (p=0.0003) (right hand figure). OS and ES tumour volume was significantly decreased in CADD522 treated mice as compared to the control.

FIG. 4 shows overall survival [%] of control vs. CADD522 treated mice with OS over time (p=0.093). Half of mice in the treatment group survived notably longer than the control group.

FIG. 5 shows metastasis-free survival [%] of control vs. CADD522 treated mice with OS over time. Markedly earlier metastasis was observed in the control group. Significantly increased metastasis-free survival period was achieved in the CADD522 treated mice with OS (p=0.0091).

FIG. 6 shows overall survival [%] of control vs. CADD522 treated mice with ES over time. Significantly increased overall survival of CADD522 treated mice with ES was observed (p=0.043).

FIG. 7 shows Micro-CT image of OS mice showing tumour associated bone disease (severe mixed effects of ectopic bone formation and lytic bone destruction) with quantification of bone volume and comparison to the non-tumour bearing contralateral (CL) leg, comparing control and CADD522 treated mice. OS tumours resulted in significantly increased bone volume in the tumour bearing leg when compared to the CL leg, whereas this bone volume difference was no longer statistically significant in CADD522 treated mice.

FIG. 8 shows Micro-CT image of ES mice showing tumour associated bone disease with comparison to the non-tumour bearing contralateral (CL) leg. Severe mixed effects of ectopic bone formation and lytic bone destruction are visible in the tumour bearing leg.

FIG. 9 shows representative H&E (Haematoxylin and Eosin) stained sections of OS tumour mass. H&E is routinely used by pathologists to stain cellular and tissue structures. OS tumours from mice treated with CADD522 were more densely packed, generally more vascularised and more organised (arranged in chords) when compared to controls. This suggests a phenotype more similar to non-cancerous bone, since bone produced from an OS tumour is of an unorganised and chaotic phenotype.

FIG. 10 shows representative H&E (Haematoxylin and Eosin) stained sections of ES tumour mass. ES tumours were generally more vascularised when treated with CADD522. This suggests a phenotype more similar to non-cancerous bone, since bone produced from an ES tumour is of an unorganised and chaotic phenotype.

### DETAILED DESCRIPTION

The presently-disclosed subject matter is illustrated by specific but non-limiting embodiments or examples throughout this description. Each example is provided by way of explanation of the present disclosure and is not a limitation thereon.

While the following terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, and materials are described.

Following long-standing patent law convention, the terms “a”, “an”, and “the” refer to “one or more” when used in this application, including the claims.

As used herein, the term “comprises” or “comprising” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”.

The features of any dependent claim may be readily combined with the features of any of the independent claims or other dependent claims. The features of any embodiment may also be readily combined with the features of any other embodiment, unless explicitly referred to or context dictates otherwise.

‘Cancer’ is one word to describe more than 200 different diseases. There are many different types of cancer that can affect different cell types and parts of the body. The origin of childhood cancer lies in aberrant human development. The spectrum of childhood cancers is mostly unique and does not have adult correlates. Adult cancers are generally of epithelial origin, arising within aging cell hierarchies as a consequence of accumulated damage and mutagenesis and increase in prevalence with age. Childhood cancers, in contrast, are overwhelmingly derived from non-epithelial lineages born in aberrantly developing tissues and display a predilection for specific post-natal age windows. Despite the success of treating some childhood cancers with cytotoxic agents, novel therapeutic strategies are required to achieve the next leap in cure rates, particularly for those childhood cancers where prognosis has remained stubbornly poor, for example osteosarcoma and Ewing sarcoma, despite intense basic biological and clinical research efforts.

Sarcomas begin in bone or soft tissues (e.g., fat, muscle or connective tissue) of the body and tend to affect children, teenagers and young adults. In contrast, carcinoma, arise in epithelial tissue, i.e., skin or tissues that line or cover internal organs and tend to affect adults.

Sarcoma accounts for 1% of all human cancer. In contrast carcinoma accounts for 99% of all human cancer.

In addition to the above differences, there are other important differences between sarcomas and carcinomas. Depending on the cell and/or tissue type, carcinomas and sarcomas have distinct karyotypes, genomes, gene expression profiles, transcriptomes and epigenomes; for example, a breast cancer (carcinoma) is littered with single nucleotide variants (SNVs) and indels whereas a bone cancer (sarcoma) will contain few SNVs and indels instead comprising complex structural alterations, e.g., translocations, chromoplexy, chromothripsis and copy number variants (CNVs). Therefore, genes and transcripts aberrantly expressed in carcinomas are not necessarily aberrantly expressed in sarcomas, e.g., tyrosine kinase genes. Hence, carcinomas and sarcomas display significantly different clinical behaviours and treatment responses, which is why each cancer is treated by a specialist oncologist specific to that cancer who spent years crafting their knowledge in the field, i.e., it is not common for one oncologist to treat several cancer types because the cancers are so different. Treatments used for carcinomas such as breast cancers colon cancers, lung cancers or hepatic cancers are very different from the treatments required for sarcomas such as bone cancers, muscle cancers, fat cancers or endothelial cancers.

Clinical trials that have attempted to treat bone sarcoma—out of desperation—using targeted breast carcinoma compounds have failed. For several examples, (i) trial NCT00001436 investigating somatostatin and tamoxifen in osteosarcoma concluded that “no sustained clinical responses were observed” (PMID: 12218590), (ii) trial NCT00023998 investigating trastuzumab in osteosarcoma concluded that “the outcome for all patients was poor, with no significant difference between the HER2-positive and HER2-negative groups . . . therapeutic benefit remains uncertain” (PMID: 22665540), (iii) trial NCT01962103 investigating Nab-paclitaxel in Ewing sarcoma showed a 0% overall response rate and concluded that “limited activity was observed” (PMID: 32554315), (iv) trial NCT00331643 investigating ixabepilone in osteosarcoma and Ewing sarcoma reported “no partial or complete responses were observed” and concluded that the drug “did not show evidence of clinical activity” (PMID: 20068084), (v) trial NCT03013127 investigating pembrolizumab in osteosarcoma was stopped early because “no patients had clinical benefit at 18 weeks of treatment and patient enrolment was stopped after completion of stage 1” (PMID: 33580363) and (vi) trial NCT04129151 investigating palbociclib in Ewing sarcoma was reported at the 2022 ASCO Annual Meeting and showed that “there were no complete or partial responders” (Shulman et al. 2022, DOI: 10.1200/JCO.2022.40.16_suppl.e23507). These several examples establish that a person skilled in the art would not perceive this approach (i.e. breast cancer drugs being effective in bone cancer) to be feasible, or worthwhile testing. The biology of breast carcinoma and bone sarcoma, as explained above, are so different there is no expectation that a treatment that works in one, would work for the other. This concept is typical knowledge in the field.

WO 2016/149667, the contents of which are incorporated herein by reference, relates to inhibitor molecules similar to those of the present invention for treating cancer. Although other cancers are mentioned, this publication primarily relates to breast cancer, and includes exemplification related to breast cancer cells only. WO 2016/149667 does not demonstrate or make plausible the effects of CADD522 on any cancer cell type other than breast cancer. There is no information or evidence to demonstrate, make plausible or teach treatment of other types of carcinomas. As discussed herein carcinomas (including breast cancer) are very different and distinct diseases from sarcomas (including OS or ES). Therefore, WO 2016/149667 provides no motivation, let alone demonstration, of treatment of sarcomas such as ES and OS. It clearly follows that a person skilled in the art could have no expectation of success that any of the treatments proposed in WO 2016/149667 would be valuable in treating any sarcomas.

Sarcomas encompass a large variety of very heterogenous cancer subtypes. Primary bone cancer (PBC) constitutes a major group of sarcomas, which itself is heterogeneous comprising many subtypes with distinct genetic drivers leading to diverse morphological features and clinical behaviour. Some of the most common sarcoma subtypes are chondrosarcoma (CS), osteosarcoma (OS) and Ewing sarcoma (ES). OS, ES and CS are discrete diseases having their own biology and clinical phenotype. Osteoblastic cells harbouring TP53 mutations give rise to OS, neural-like cells with an EWSR1::FLI1 fusion give rise to ES and chondroblastic cells with IDH1/2 mutations are associated with some CS cases, however, the CS driver mutation remains unknown.

While all three of the above mentioned PBC subtypes are distinct diseases with distinct genetic drivers and distinct clinical behaviour and treatment response, the following features are common to OS and ES, and distinguish them from CS. OS and ES are most common in children, teenagers and young adults and sporadic OS and ES are almost never diagnosed in individuals over the age of 30 years. In contrast, CS arises more prevalently in adults and sporadic CS is almost never diagnosed in individuals under the age of 40 years. OS and ES are high-grade at diagnosis and harbour an accelerated propensity for metastasis. Unlike OS and ES, CS presents relatively distinguishable histology between low-grade, intermediate-grade and high-grade at diagnosis. The underlying molecular biology is significantly different for ES and OS in comparison with CS. Hence, methods of treatment and therapeutics that are used for treating CS in adults are not successful in treating childhood PBCs such as OS and ES. As far as the inventors are aware, there are currently no licenced or patented therapies with high effectiveness in OS and ES. Trials investigating treatments for OS and ES that showed some success, which treatments were then tested in CS failed. For example, trial NCT01267955 (Vismodegib). This demonstrates that the diseases are distinct, biologically different and differentially clinically managed and therefore cannot be and are not assumed to respond similarly to treatments.

WO 2020/128534, the contents of which are incorporated herein by reference, relates to inhibitors similar to those of the present invention in order to treat chondrosarcoma. As discussed herein chondrosarcoma is a very different disease from OS or ES and therefore a person skilled in the art could have no expectation of success that any of the treatments proposed in WO 2020/128534 would be valuable in treating OS or ES prior to the present invention. CS cells, which are similar to cartilage cells, are entirely different from OS and ES cells, which are similar to bone cells. In fact, CS cells stem from cells with in-built and inherent mechanisms to specifically prevent becoming bone-like (e.g., SOX9 and IHH signalling). This completely changes the cell morphology and behaviour of CS cells as compared to OS and ES cells.

An in-depth review of the distinctive biology of cancer in adolescents and young adults found that the biology of the cancers in this age group is different from that in other age groups, not only in the spectrum of cancers but also within individual cancer types. It has been concluded that researchers should not assume that the biology of cancers is the same in the different age groups.

ES and OS being high-grade at diagnosis hampers research efforts to find new therapeutic targets and/or treatments because cancer progression from low-grade to high-grade cannot be observed. Examination of such progression may usually provide clues and/or a better understanding of fundamental events plus metastatic hallmarks in high-grade OS and ES, and result in the identification of new treatment options.

Current treatment involves non-specific combination chemotherapy with surgery. Surgery is highly invasive and risks incomplete removal of the affected tissue as well as causing metastatic spread. Chemotherapy is often associated with complex and severe side effects because it generally targets rapidly dividing cells. As children are still growing and many kinds of healthy cells are dividing faster than in adults, chemotherapy during childhood can damage these cells leading to unwanted long-term effects on growth and development. Unfortunately, neither OS nor ES have seen treatment or survival improvement for decades. Therefore, new effective treatment options for ES and OS are urgently needed.

Most ES and OS cancer deaths are due to metastatic disease. The composition of the invention was found to specifically increase metastasis free survival in mice, suggesting that the treatment may be especially effective to prevent metastasis, and/or treat metastatic OS and ES in children, therefore increasing survival.

The standard method of treatment for ES and OS is cytotoxic chemotherapy, which targets all rapidly dividing cells, including healthy cells. In contrast, the present inventors have now provided a new therapy which targets the activity of a specific molecular target. The therapy of the present invention targets RUNX2, which is aberrantly expressed in ES and OS cells. It was surprisingly found that there is increased expression of RUNX2 in OS and ES. RUNX2 would normally promote differentiation of normal bone cells. Therefore, it is unexpected that RUNX2 would support the cancerous phenotype of ES and OS cells. Even more surprisingly, inhibition of RUNX2 binding to DNA leads to significant reduction in tumour volume in vivo, and increased survival and reduced metastasis in mouse OS and ES models. Notably, prior to the present invention it was well understood in the field of cancer therapy that RUNX2 cannot be considered a therapeutic target in OS. Therefore, the state of the art in cancer therapy teaches away from any treatment options for OS and ES involving inhibition of RUNX2.

The present invention provides a composition for use in a method of treating osteosarcoma, Ewing sarcoma and/or a metastatic cancer originating from osteosarcoma or Ewing sarcoma, wherein the composition comprises a compound according to formula (I) or a pharmaceutically acceptable salt, ester, derivative or prodrug thereof.

The present invention also provides a method of treating a cancer selected from the group consisting of osteosarcoma, Ewing sarcoma, a metastatic cancer originating from an osteosarcoma and a metastatic cancer originating from Ewing sarcoma, the method comprising administering to a subject in need thereof a composition comprising a compound according to formula (I) or a pharmaceutically acceptable salt, ester, derivative or prodrug thereof.

The present invention also provides a use of a composition in the manufacture of a medicament for the treatment of osteosarcoma, Ewing sarcoma and/or a metastatic cancer originating from osteosarcoma or Ewing sarcoma, wherein the composition comprises a compound according to formula (I) or a pharmaceutically acceptable salt, ester, derivative or prodrug thereof.

The present invention also provides a use of a composition to treat osteosarcoma, Ewing sarcoma and/or a metastatic cancer originating from osteosarcoma or Ewing sarcoma, wherein the composition comprises a compound according to formula (I) or a pharmaceutically acceptable salt, ester, derivative or prodrug thereof.

### Formula I

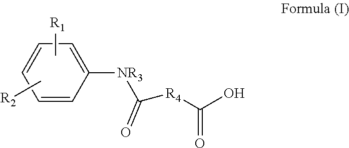

wherein R₁ and R₂ are each independently selected from hydrogen, a halogen (e.g. Cl), an alkyl (e.g. a C₁₋₃ alkyl straight chain alkyl or a C₅₋₇ cycloalkyl), a haloalkyl, an alkylamide, a cycloalkylamide or an alkyamine (e.g. a dialkylamine);
R₃ is H or alkyl; and
R₄ is a bridged cycloalkenyl ring (e.g. cyclohexene ring with an alkyl bridge such as a methylene bridge which may be optionally substituted, for example optionally substituted with a cycloalkyl ring such as a C₃₋₆ cycloalkyl ring (e.g. a cyclopropane ring)). The alkyl moiety may, for example, be a C₁₋₃ alkyl chain. The cycloalkyl moiety may, for example, be a C₅₋₇ ring.

In certain embodiments, R₁ and R₂ are each independently selected from H, Cl, F, Br, CH₃, CF₃, SH, —N(C₁₋₃alkyl)₂, —NHC(O)C₁₋₃alkyl, and —NHC(O)C₅₋₇cycloalkyl.

In certain embodiments, R₃ is H or C₁₋₃ alkyl.

In certain embodiments, R₄ is

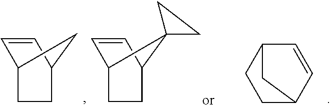

R₄ may be bonded to the adjoining atoms in the compound of formula (I) at the positions indicated by the dashed lines,

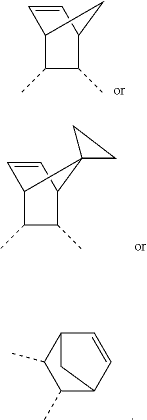

In certain embodiments, R₃ is H.

In certain embodiments, R₁ and R₂ are each individually selected from H, Cl, Br and —NHC(O)CH₃, R₃ is H, and R₄ is

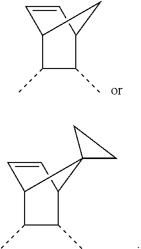

In certain embodiments, R₁ and R₂ are each individually selected from H, Cl, CH₃, —NHC(O)CH₃, —NHC(O)cyclohexane, or —N(CH₃)₂, R₃ is H and R₄ is

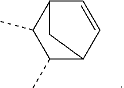

The compound according to formula (I) may, for example, be 3-(N-(3,4-dichlorophenyl)carbamoyl)-5-norbornene-2-carboxylic acid, also named 3-{[(3,4-dichlorophenyl)amino]carbonyl}bicyclo[2.2.1]hept-5-ene-2-carboxylic acid, known as CADD522 (CAS No: 199735-88-1),

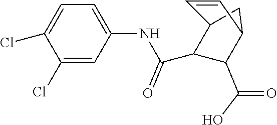 [SMILES: O=C(O)C1C2C=CC(C2)C1C(=O)Nc1ccc(Cl)c(Cl)c1]

or a salt, ester, derivative or prodrug thereof, preferably a pharmaceutically acceptable salt, ester, derivative or prodrug thereof.

Alternatively, the compound according to formula (I) may, for example, be one or more selected from the following:

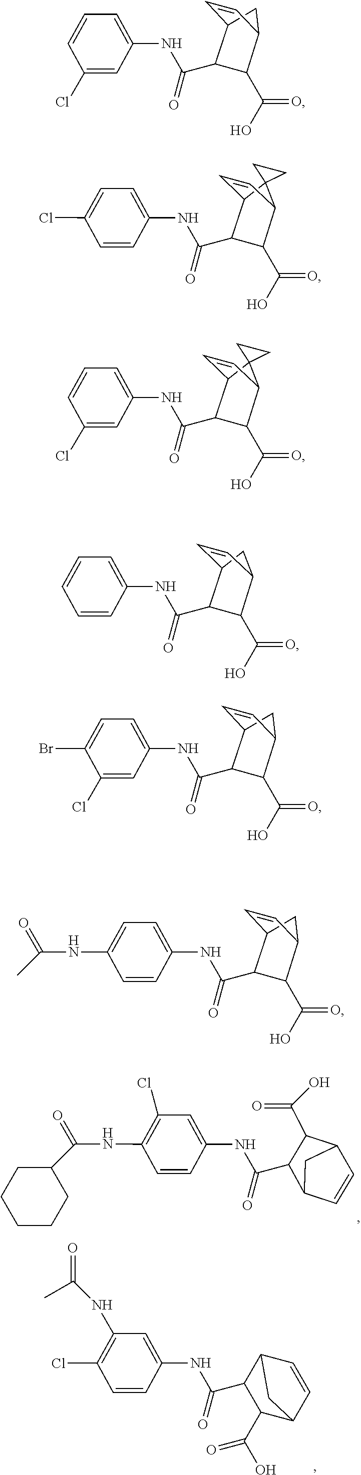

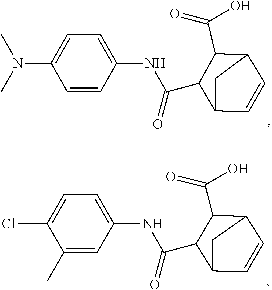 [SMILES: CN(C)c1ccc(NC(=O)C2C3C=CC(C3)C2C(=O)O)cc1 ; Cc1cc(NC(=O)C2C3C=CC(C3)C2C(=O)O)ccc1Cl]

and salts, esters, derivatives and prodrugs thereof, for example pharmaceutically acceptable salts, esters, derivatives and prodrugs thereof.

The compositions used in the methods of treatment described herein may, for example, be pharmaceutical compositions. The term “pharmaceutical composition” refers to a composition comprising (a pharmaceutically effective amount of) a therapeutic active agent (i.e., a compound of formula (I)). The pharmaceutical compositions disclosed herein may additionally comprise one or more pharmaceutically acceptable carriers and/or excipients and/or diluents. The phrase “pharmaceutically acceptable” refers to compositions that do not produce an adverse, allergic or other untoward reaction when administered to an animal, such as, for example, a human.

The compositions and pharmaceutical compositions disclosed herein may further contain ingredients selected from, for example, adjuvants, carriers (solvents) such as water, ethanol, polyols (e.g. glycerol, propylene glycol, liquid polyethylene glycol), lipids, and combinations thereof, preserving agents, stabilisers, fillers, binders, disintegrating agents, wetting agents, emulsifying agents, suspending agents, sweetening agents, flavouring agents, perfuming agents, lubricating agents, coating agents, encapsulating agents and dispersing agents, depending on the nature of the mode of administration and dosage forms.

The compositions and pharmaceutical compositions disclosed herein may take the form, for example, of a solid preparation including tablets, capsules, caplets, drageés, lozenges, granules, powders, pellets, beads, cachets and bolus; and a liquid preparation including elixir, syrups, suspension, spray, emulsion, lotion, solution or tincture; or a semi-solid preparation including ointment, cream, paste, gel or jelly. Also included are solid form preparations, for example, tablets, capsules, granules and powder, which are intended to be converted, shortly before use, to liquid form preparations for oral administration. Techniques and formulations generally may be found in Remington, The Science and Practice of Pharmacy, Mack Publishing Co., Easton, PA, latest edition.

Any suitable mode of administration may be used. For example, the administration may be oral, local, parenteral (including intravenous, intramuscular, subcutaneous and intradermal), transdermal (including percutaneous) or transmucosal (including inhalation, nasal and sublingual). The administration may be sufficient to contact the tumour cells with the compound of formula (I).

The composition may, for example, be administered to the subject for any period of time suitable to obtain a desired result (e.g., treatment of the OS, ES or metastatic cancer originating from OS or ES). The composition may, for example, be administered at any frequency suitable to obtain the desired result, for example daily or weekly or monthly. This includes intermittent and/or repeated administration.

### Examples

### Materials and Methods

Cell lines. 143B (human OS) and TC71 (human ES) cells were obtained from ATCC. Cells were authenticated by STR profiling and were cultured in DMEM/F-12 (Thermo Fisher Scientific) or IMDM (Thermo Fisher Scientific) containing 1% (v/v) insulin transferrin selenium, 10% (v/v) FBS and 1% (v/v) penicillin/streptomycin. Medium was refreshed every other day and cells were maintained at 37° C. in 5% CO₂. For the animal experiments 143B and TC71 cells were modified by transfecting with the GFP-luciferase tagged LVP02 lentivirus (AMSBIO). Adding the luciferase tag had no effect on cell phenotype.

Proliferation assays. 1×10³ cells in 100 μL of medium were seeded per well into 96-well plates. PBS was added to outer wells to prevent evaporation. All plates were incubated for 24 h at 37° C. in 5% CO₂. 100 nM, 1 μM, 10 μM and 100 μM CADD522 were added from a stock concentration of 10 mM in DMSO and incubated for 72 h before measuring cell viability/proliferation using the WST-1 assay kit (Abcam) following manufacturer's instructions. Data was presented as relative to the control (appearing as −11 on x-axis), which is set to 100% viability. Experiments were performed in quadruplicates in three independent experiments.

Mice. The University of Sheffield Animal Welfare and Ethics Committee approved the animal experiments. Experiments were performed under licence in accordance with the UK Home Office guidelines and under the Animals (Scientific Procedures) Act 1986. Six- to seven-week-old female BALB/c nude mice (Charles River Laboratories) were acclimatised for one week prior to in vivo study. Mice were housed with a 12 h light-dark cycle at 22° C. and had free access to 2018 Teklad global 18% protein rodent diet containing 1.01% calcium (Harlan) and water. Mice were anesthetised by isoflurane inhalation before implantation with 2.5×10⁵ 143B+GFP-Luc (OS) cells or 5×10⁵ TC71+GFP-Luc (ES) cells in 20 μl PBS onto the tibial surface. Mice were matched by weight and initial IVIS signal and then randomly divided into groups (n=8 mice/group). CADD522 treatment commenced the day after tumours became visible. Mice received either vehicle control (PBS and DMSO) or CADD522 (25 mg/kg) by IP injection five times per week. Tumours were measured by callipers throughout the study twice per week blinded. Two perpendicular measurements were taken and volume calculated by the formula V=0.523×L×(S)² where L and S refer to the largest and smallest measurement. Mice were treated until the palpable tumour reached the permissible 12 mm diameter limit. At the end of the procedure ex vivo imaging of mouse internal organs was performed to detect metastasis. Prior to killing, mice were injected subcutaneously with 150 mg/kg XenoLight D-Luciferin substrate (Perkin Elmer) that was left to disperse for a minimum of 5 m. Dissected organs were then imaged using the IVIS Lumina II. Mice legs and lungs were dissected and fixed in 10% formalin for further analysis.

Micro-CT. Fixed tibiae were scanned using a SkyScan 1172 desktop micro-CT machine (Bruker) at 8 um resolution with the X-ray source operating at 50 kV, 200 uA and using a 0.5 mm aluminium filter. Images were captured every 0.7°. Scanned images were reconstructed using Skyscan NRecon software v1.6.9 (Bruker). The region of interest (ROI) for the total bone volume was selected to include both the tibia and fibula and was determined at the top of the bone as soon as the tibia enters the image to the lower point where the tibia and fibula meet.

Histology. Bones were fixed in neutral buffered formalin for 48 h after which they were transferred to 70% ethanol. Bones were then decalcified in 10% EDTA, embedded in paraffin and 5 μm sections produced. Sections were de-waxed in xylene, rehydrated through graded alcohols and stained in haematoxylin (VWR) and eosin. The sections were dehydrated through graded alcohols and coverslips were mounted in DPX and imaged using Olympus BX51 microscope (Olympus Life Sciences) and Pannoramic 250 Flash III slide scanner (3DHISTECH).

Statistics. For statistical tests p=<0.05 was considered as statistically significant. Proliferation data is reported for each experimental repeat (n=3). Data was tested for normality by D'Agostino Pearson Omnibus test and analysed in GraphPad Prism v9 with p=<0.05 regarded as significant. A one-way ANOVA was used to identify any differences in proliferation across the concentration range tested compared to the untreated control. A post-hoc Dunnett's multiple comparison was then used to determine which concentration impacted proliferation compared to the untreated control. Analysis was performed for each independent experiment and for the average results across experiments for each cell line and exposure period. Analysis was performed for each independent experiment and for the average results across experiments for each cell line and exposure period. Prism (v9) (GraphPad) was also used to generate the survival curves, with a Log-rank (Mantel-Cox) test used to determine if the survival rates were different between groups. Metastasis-free survival data was generated using the length of time before the mouse had to be killed due to metastasis-associated adverse effects. Overall survival data was generated using the length of time before the mouse had to be killed due to adverse effects irrespective of metastasis.

### Results

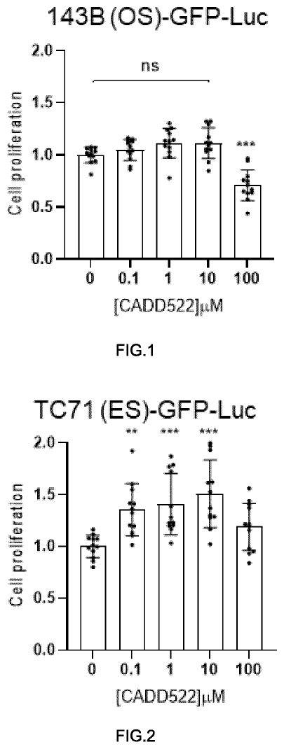

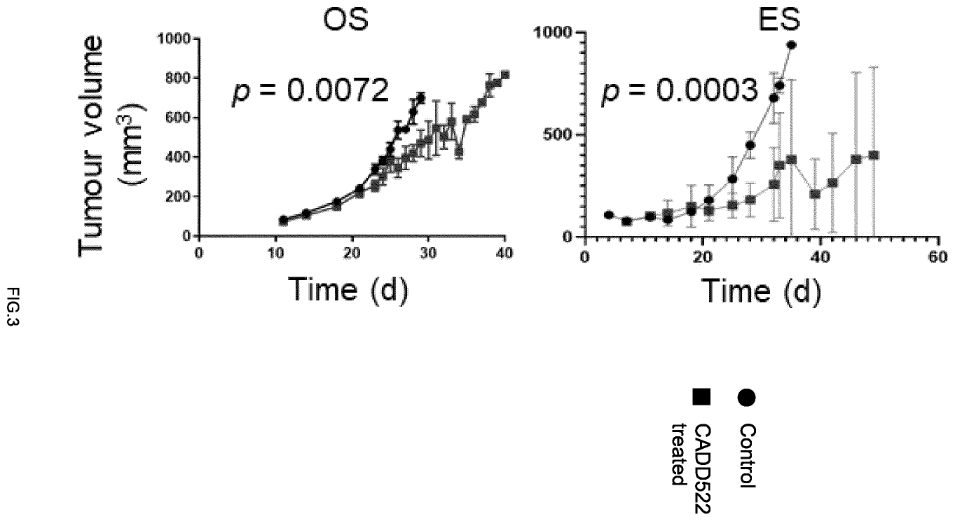

To determine if RUNX2 pharmacological regulation would prove to be a potential therapeutic option in OS/ES the novel compound computer aided drug design molecule 522 (CADD522) (C₁₅H₁₃Cl₂NO₃) was synthesized. CADD522 effect on OS/ES cell proliferation was assessed in vitro. OS cells showed significantly reduced cell proliferation after 100 μM CADD522 treatment for 72 h (FIG. 1). Additionally, ES also showed cell proliferation differences after 100 μM CADD522 treatment for 72 h (FIG. 2).

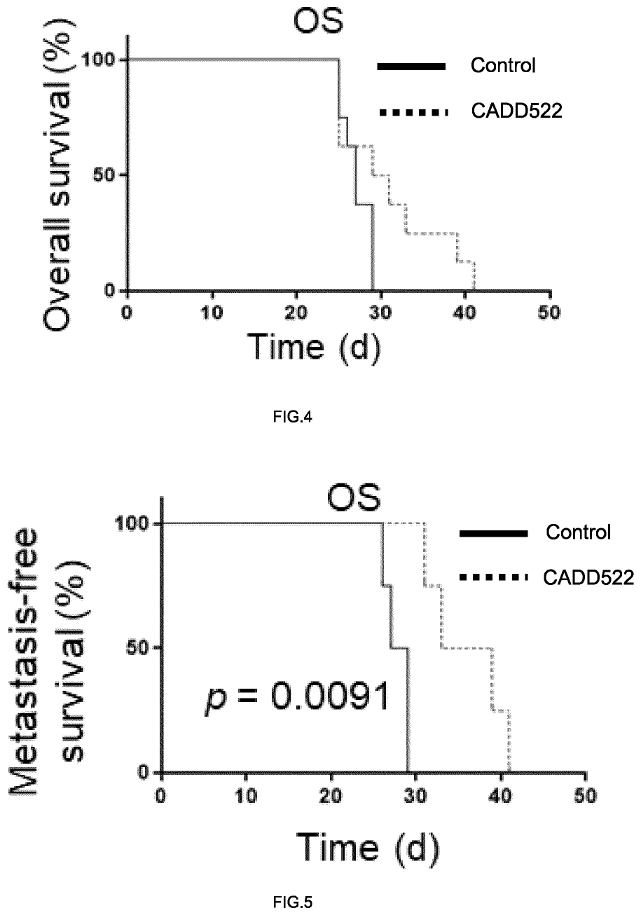

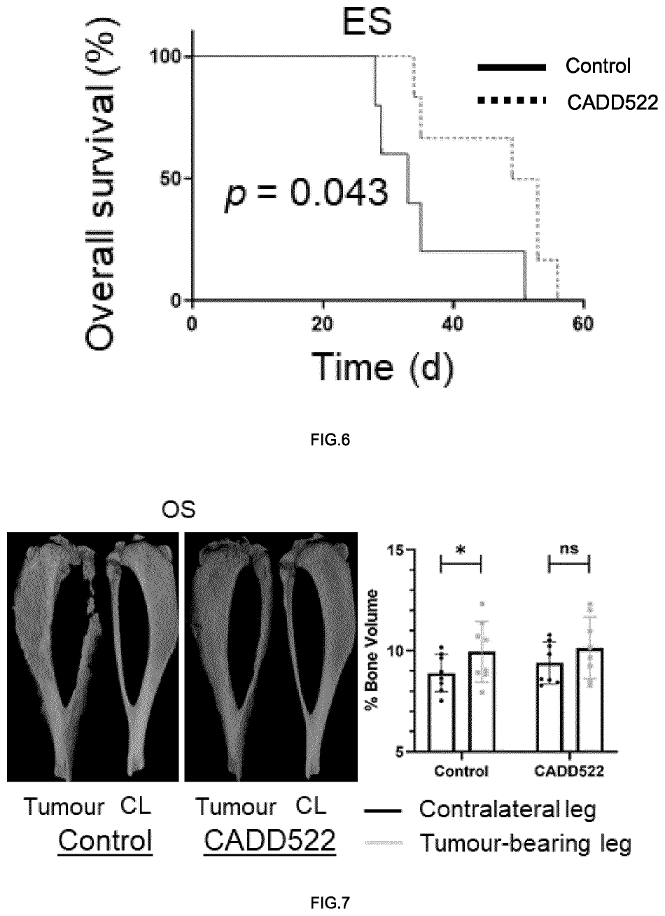

CADD522 was assessed in vivo using a PBC xenograft mouse model. CADD522 significantly reduced tumour volume over time in both OS (p=0.0072) and ES (p=0.0003) models (FIG. 3). Half of OS mice receiving CADD522 treatment survived longer than the control group OS animals (FIG. 4). There was also markedly earlier metastasis in the control group as compared to the CADD522 treatment group (FIG. 5). CADD522 treatment showed significantly increased metastasis-free survival in OS animals (p=0.0091) (FIG. 5). Significantly increased overall survival with CADD522 treatment in ES animals was observed (p=0.043) (FIG. 6). The inventors predict that the results would be even more significant in humans, because, unlike in mice, treatment usually includes the surgical removal of the primary tumour tissue.

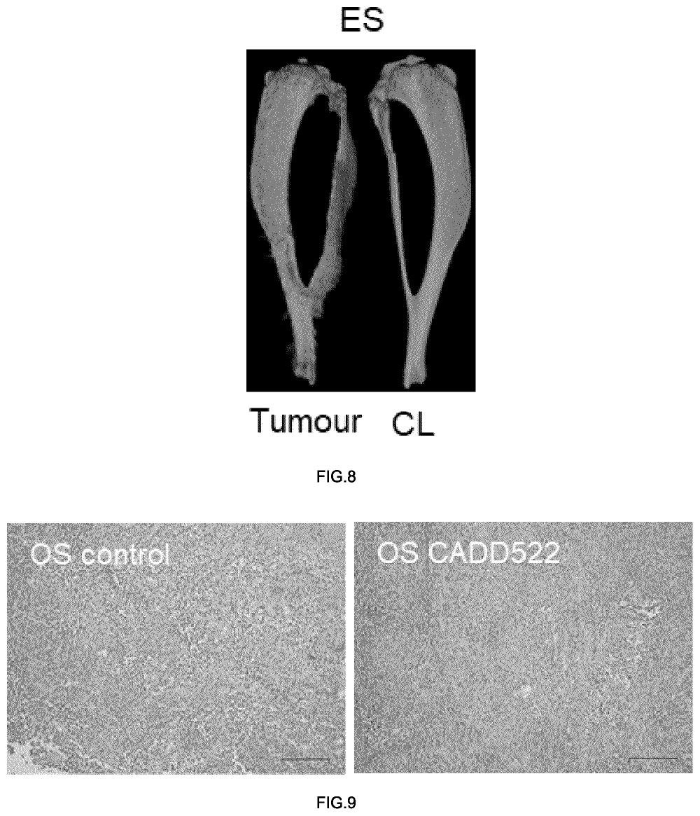

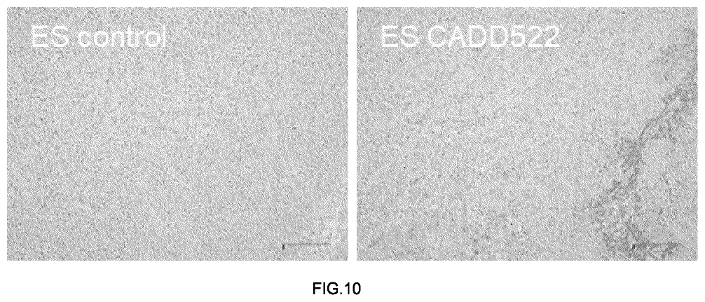

PBC clinical complications can include cancer-associated bone disease that manifests as ectopic osteoid/cartilage matrix and/or lytic destruction. CADD522 effect on bone disease observed in these models was assessed by microCT. When comparing the effect of OS tumours on bone volume, control mice presented with severe mixed effects including ectopic bone formation and lytic bone destruction (FIG. 7). OS tumours resulted in an overall significantly increased bone volume in the tumour bearing leg when compared to the non-tumour bearing contralateral (CL) leg (p=0.02) (FIG. 7). In CADD522 treated mice this bone volume difference was no longer statistically significant suggesting less severe cancer-associated bone disease (FIG. 7). ES tumours demonstrated a similar phenotype with visible ectopic bone formation and lytic destruction (FIG. 8). Histological examination of the tumours at endpoint revealed that OS tumours from mice treated with CADD522 were more densely packed, generally more vascularised and more organised (arranged in chords) when compared to controls that also comprised more bony islands (FIG. 9). ES tumours showed a similar pattern of being generally more vascularised when treated with CADD522 (FIG. 10).

### DISCUSSION

From the anatomy affected and the treatments employed childhood cancer (mostly blood, brain/central nervous system, sarcoma) is fundamentally dissimilar to adult cancer. The latter and more common cancers arise from decades of mutagenesis; ‘hot’ tumours littered with single nucleotide variants (SNVs) and indels. Childhood cancer drivers are less well understood but the malignant genomes are recognised to be quiet and ‘cold’ instead comprising structural variants (SVs) and copy number variants (CNVs). SVs/CNVs may be the final step in overt transformation as recent and emerging evidence suggests childhood cancers begin as developmental errors. Some childhood cancers will not be preventable meaning the best strategy to approach childhood cancer is early and accurate diagnosis followed by effective and gentle treatment. Conventional OS and ES are high-grade at diagnosis making it difficult to elucidate early metastatic events that occur immediately or soon after driver events but before evolution has significantly progressed and made it almost impossible to untangle drivers and passengers. Therefore, identification of therapeutic treatments based on underlying disease mechanisms has been extraordinarily challenging. Surprisingly and unexpectedly, the present study identifies CADD522 as promising candidate for treatment of OS and ES.

Pre-clinical assessment of CADD522 demonstrated effects on all PBC models tested both in vitro and in vivo. CADD522 proved to be cytotoxic to OS and ES cells in vitro, and PBC mouse models treated with the compound demonstrated reduced tumour size, increased metastasis-free survival in OS as well as reduced cancer-associated bone disease in vivo. There was no observed toxicity in the animals when treated with 25 mg/kg over the study period.

1. A composition for use in a method of treating osteosarcoma, Ewing sarcoma and/or a metastatic cancer originating from osteosarcoma or Ewing sarcoma, wherein the composition comprises a compound according to formula (I) or a pharmaceutically acceptable salt, ester, derivative or prodrug thereof, 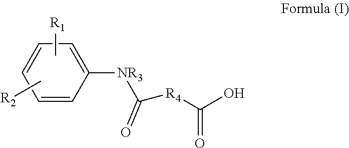
wherein R₁ and R₂ are each independently selected from hydrogen, a halogen, a haloalkyl, an alkyl, an alkylamide, a cycloalkylamide or an alkyamine;
R₃ is H or alkyl; and
R₄ is a bridged cycloalkenyl ring.

2. A method of treating a cancer selected from the group consisting of osteosarcoma, Ewing sarcoma, a metastatic cancer originating from an osteosarcoma and a metastatic cancer originating from Ewing sarcoma, the method comprising administering to a subject in need thereof a composition comprising a compound according to formula (I) or a pharmaceutically acceptable salt, ester, derivative or prodrug thereof, 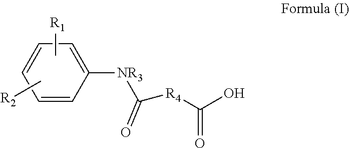
wherein R₁ and R₂ are each independently selected from hydrogen, a halogen, a haloalkyl, an alkyl, an alkylamide, a cycloalkylamide or an alkyamine;
R₃ is H or alkyl; and
R₄ is a bridged cycloalkenyl ring.

3. (canceled)

4. Use of a composition to treat osteosarcoma, Ewing sarcoma and/or a metastatic cancer originating from osteosarcoma or Ewing sarcoma, wherein the composition comprises a compound according to formula (I) or a pharmaceutically acceptable salt, ester, derivative or prodrug thereof, 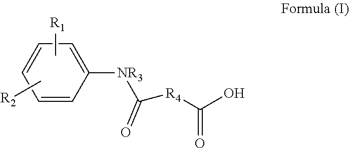
wherein R₁ and R₂ are each independently selected from hydrogen, a halogen, a haloalkyl, an alkyl, an alkylamide, a cycloalkylamide or an alkyamine;
R₃ is H or alkyl; and
R₄ is a bridged cycloalkenyl ring.

5. The method of claim 2, wherein metastasis of the osteosarcoma or Ewing sarcoma is inhibited.

6. The method of claim 2, wherein the osteosarcoma or Ewing sarcoma overexpresses RUNX2 in comparison to normal bone tissue or other biological material.

7. The method of claim 2, wherein R₁ and R₂ are each independently selected from H, Cl, F, Br, CH₃, CF₃, SH, —N(C₁₋₃alkyl)₂, —NHC(O)C₁₋₃alkyl, and —NHC(O)C₅₋₇cycloalkyl.

8. The method of claim 2, wherein R₃ is H or C₁₋₃ alkyl.

9. The method of claim 2, wherein R₄ is

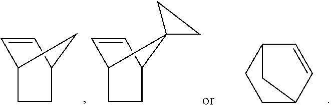

10. The method of claim 2, wherein the compound of formula (I) is 3-(N-(3,4-dichlorophenyl)carbamoyl)-5-norbornene-2-carboxylic acid, also named 3-{[(3,4-dichlorophenyl)amino]carbonyl}bicyclo[2.2.1]hept-5-ene-2-carboxylic acid, known as CADD522 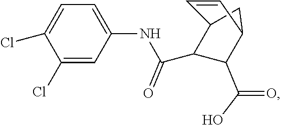 [SMILES: O=C(O)C1C2C=CC(C2)C1C(=O)Nc1ccc(Cl)c(Cl)c1]
or a salt, ester, derivative or prodrug thereof.
